(19) United States
(12) Patent Application Publication
(10) Pub. No.: US 2007/0142752 A1
(43) Pub. Date: Jun. 21, 2007

(54) Uterine cavity length measurement

(76) Inventors: Robert Kotmel, Burlingame, CA (US); J. Brook Burley, Sunnyvale, CA (US); Russel M. Sampson, Palo Alto, CA (US); Estela H. Hilario, Los Altos, CA (US)

Correspondence Address: FISH & RICHARDSON P.C., PO BOX 1022, MINNEAPOLIS, MN, 55440-1022, US

(21) Appl. No.: 11/313,444

(22) Filed: Dec. 20, 2005

(51) Int. Cl. A61B 5/103

(52) U.S. Cl. 600/591

(57) ABSTRACT

A uterine length measurement device includes a first elongate member having a distal end, a proximal end, and a lumen, the elongate member configured for insertion into an endocervical canal where the distal end is configured for insertion to approximately an internal cervical os of the endocervical canal. A second elongate member is provided having a distal end and a proximal end, the second elongate member configured to move within the lumen of the first elongate member, where the distal end can protrude from the distal end of the first elongate member and is configured for insertion to approximately the fundus of a uterine cavity. Moving the second elongate member relative to the first elongate member to position the distal end of the second elongate member at approximately the fundus of the uterus provides a direct measurement of a length of the uterine cavity.

### TECHNICAL FIELD

This invention relates to medical devices and techniques.

### BACKGROUND

The human uterine cavity is approximately triangular in shape and relatively flat, much like an envelope. The cavity is entered via the endocervical canal. The proximal end of the canal, the external cervical os, opens to the vagina while the distal end, the internal cervical os, opens to the uterine cavity. The tip of the triangular-shaped uterine cavity is located at the internal cervical os, while the base is defined by the openings that lead to the fallopian tubes, the tubal ostia. Sounding the uterus, i.e., determining the length from the fundus of the uterine cavity to the external cervical os (referred to herein as the “sounding length”), is usually a blind procedure. A physician inserts a uterine sound transcervically and advances the sound until it reaches the fundus. The length from the fundus to the external cervical os can be measured directly using graduations stamped on the shaft of the sound. The physician relies upon tactile feedback to determine when the uterine sound has reached the fundus.

Conventional uterine sounds are constructed from a malleable metal material, approximately 3.5 mm in diameter with a working length of roughly 25 cm, and have a flattened handle portion the physician can grasp. The uterine sound necessarily is substantially rigid in the axial direction and somewhat flexible out of plane, transverse to its axis, in order to reach the fundus and provide the physician the tactile sensation of touching the fundus.

Conventional uterine sounds provide a direct measurement of the sounding length, which includes both the uterine cavity length and the endocervical canal length. However, a physician may need to know the uterine cavity length alone in order to perform certain medical procedures. Conventional uterine sounds do not provide direct measurement of the uterine cavity length without additional measurements or other calculations.

The details of one or more embodiments of the invention are set forth in the accompanying drawings and the description below. Other features, objects, and advantages of the invention will be apparent from the description and drawings, and from the claims.

### SUMMARY

In general, in one aspect, a uterine length measurement device is provided. The uterine length measurement device includes a first elongate member having a distal end, a proximal end and a lumen. The elongate member is configured for insertion into an endocervical canal, where the distal end is configured for insertion to approximately an internal cervical os of the endocervical canal. The uterine length measurement device also includes a second elongate member having a distal end and a proximal end. The second elongate member is configured to move within the lumen of the first elongate member. The distal end can protrude from the distal end of the first elongate member and is configured for insertion to approximately the fundus of a uterine cavity. The device is configured such that positioning the distal end of the first elongate member at approximately the internal cervical os and moving the second elongate member relative to the first elongate member to position the distal end of the second elongate member at approximately the fundus of the uterus provides a direct measurement of a length of the uterine cavity.

Implementations can include one or more of the following features. The first elongate member can further include a stepped portion at the distal end configured to facilitate locating the internal cervical os. The first elongate member can further include graduations marked on at least a portion of a length of the first elongate member for indicating relative movement of the second elongate member to the first elongate member, the relative movement corresponding to the direct measurement of the length of the uterine cavity. The first elongate member can also include second graduations marked on a second portion of the length of the first elongate member for measuring a length of the endocervical canal.

The uterine length measurement device can further include a control knob located near the proximal end of the second elongate member and a slot formed in proximal region of the first elongate member and adjacent to the graduations and configured to receive the control knob. The control knob can be movable within the slot to advance and retract the second elongate member within the lumen of the first elongate member and the position of the control knob relative to the graduations when the distal end of the second elongate member is positioned at approximately the fundus of the uterus indicates the length of the uterine cavity. The uterine length measurement device can further include a collar configured to move along the first elongate member to mark a position relative to the second graduations identifying a measurement of the endocervical canal. The distal end of the second elongate member can include an a traumatic tip. The a traumatic tip can include a concave region configured to collect endometrial tissue when the a traumatic tip is positioned within the uterine cavity and retracted toward the proximal end of the first elongate member.

The second elongate member can be substantially rigid compressively between the distal and proximal ends, and substantially flexible out of a plane of a longitudinal axis of the second elongate member. Additionally, the second elongate member can be substantially flexible along a first plane out of a longitudinal axis of the second elongate member and substantially rigid along a second plane out of the longitudinal axis. The flexibility of the second elongate member varies from the proximal to the distal end such that the distal end has a greater flexibility than the proximal end.

The uterine length measurement device can further include one or more locking grooves in the first elongate member configured to hold the control knob in place relative to the first elongate member when the control knob is rotated into a particular locking groove. The uterine length measurement device can further include a locking collar coupled to the control knob, the locking collar configured to tighten around the first elongate member to lock the control knob relative to the first elongate member. The distal tip of the second elongate member can be atraumatic and can be a full radius tip, a chamfered tip, or a convex tip.

In general, in one aspect, a uterine length measurement device is provided. The uterine length measurement device includes a first elongate member having a distal end, a proximal end and a lumen, the elongate member configured for insertion into an endocervical canal where the distal end is configured for insertion to approximately an internal cervical os of the endocervical canal. The uterine length measurement device also includes a second elongate member having a longitudinal axis, a distal end and a proximal end. The second elongate member is configured to move within the lumen of the first elongate member. The distal end can protrude from the distal end of the first elongate member and is configured for insertion to approximately the fundus of a uterus. An end cap is connected to the distal end of the second elongate member, the end cap being in a closed position when the second elongate member is inserted into the uterus and switching into an open position when a force applied to a distal tip of the end cap by the uterus exceeds a threshold force. A surface area of the end cap projected onto a plane substantially perpendicular to the longitudinal axis of the second elongate member is enlarged in the open position as compared to in the closed position. The open position resists penetration of the end cap into a wall of the uterus. The device is configured such that positioning the distal end of the first elongate member at approximately the internal cervical os and moving the second elongate member relative to the first elongate member to position the distal end of the second elongate member at approximately the fundus of the uterus provides a direct measurement of a length of a uterine cavity.

Implementations can include one or more of the following features. The end cap can include one or more fin members, where the fin members are positioned along a longitudinal axis of the elongate member when the end cap is in the closed position and deploy laterally from the longitudinal axis when switching into the open position. The uterine length measurement device can further include a deployment mechanism, configured to switch the end cap from the closed position to the open position upon a force on the distal tip of the end cap exceeding the threshold force. The end cap can include one or more fin members, where the fin members are positioned along a longitudinal axis of the elongate member when the end cap is in a closed position and deploy laterally from the longitudinal axis when switching into the open position. The elongate member can include a rod connected to the distal tip of the end cap. The deployment mechanism can include a spring positioned about the elongate member, the spring preloaded to exert the threshold force on a first face of a retainer connected to the rod, where the threshold force exerted by the spring prevents the rod from translating in a direction away from the end cap. The retainer can include a second face abutting a housing preventing translation of the rod in a direction toward the end cap. When a force on the distal end of the end cap exceeds the threshold force, the rod can translate axially compressing the spring and thereby translating the distal end of the end cap causing the one or more fin members to deploy laterally switching the end cap into the open position. The uterine length measuring device can further include an indicator configured to provide an indication to a user when the end cap has switched from the closed to the open position.

In general, in one aspect, a method for using a uterine measurement device is provided. The method includes transcervically inserting the measurement device including advancing a first elongate member of the device to approximately the internal cervical os, advancing a second elongate member of the device, located within the first elongate member, relative to the first elongate member to an internal fundus of the uterine cavity. The uterine cavity length is measured according to the position of the second elongate member relative to the first elongate member.

Implementations can include one or more of the following features. The method can further include automatically locating the internal cervical os according to a feature positioned at the distal end of the first elongate member. Locating can include receiving tactile feedback from the feature contacting the internal cervical os. The feature can be a step feature, a balloon feature, or a groove feature. The method can further include sliding a control knob along the first elongate member to advance the second elongate member and locking the control knob to hold the second elongate member in place relative to the first elongate member. Locking the control knob can include rotating the control knob into a groove in the first elongate member or rotating a slidable collar attached to the control knob, where rotating the slidable collar tightens the collar around the first elongate member to hold the control knob in place. The method can further include moving the collar along the first elongate member to the external cervical os to measure the cervical canal length according to the position of the movable collar relative to the first elongate member.

Implementations of the devices and methods described herein can provide one or more of the following advantages. A uterine measurement device can be provided such that a user can directly measure the length of just the uterine cavity. Additionally, the length of the endocervical canal can also be directly measured.

The uterine measurement device can include an atraumatic tip at the distal end to prevent trauma, including perforation of the uterine wall, during a uterine length measurement procedure. The atraumatic tip can also include a collection cup for taking a tissue sample from the uterine cavity wall. The uterine measurement device can include a lockable control knob that allows the user to lock the position of the inner member of the uterine measurement device in place, relative to the outer sheath, for later reading of the length measurement.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an isometric view of a uterine cavity length measurement device.

FIG. 2A shows a side view of a uterine cavity length measurement device in a retracted position.

FIG. 2B shows a side view of a uterine cavity length measurement device in an extended position.

FIG. 3 is a flowchart showing a process for directly measuring the length of uterine cavity.

FIG. 4 shows a portion of a member of a uterine cavity length measurement device.

FIG. 5 shows an implementation of a collection cup coupled to a tip of a member of a uterine cavity length measurement device.

FIG. 6 shows an isometric view of a uterine cavity length measurement device including a lockable control knob.

FIG. 7 shows an isometric view of a uterine cavity length measurement device including locking grooves.

FIG. 8A shows side and end views of a full radius tip of a uterine measurement device.

FIG. 8B shows side and end views of a chamfered tip of a uterine measurement device.

FIG. 8C shows side and end views of a concave tip of a uterine measurement device.

FIG. 9A shows a full radius tip of a uterine measurement device producing an axial load on the uterine wall.

FIG. 9B shows a chamfered tip of a uterine measurement device producing an axial load on the uterine wall.

FIG. 9C shows a concave tip of a uterine measurement device producing an axial load on the uterine wall.

FIG. 10A is a top view of an inner member of a uterine measurement device in a closed position.

FIG. 10B is a top view of the inner member of FIG. 10A in an open position.

FIG. 11 is a top view of the end cap of the inner member of FIG. 10A.

FIG. 12 is a top view of the end cap of the inner member of FIG. 10B.

FIG. 13 is a cutaway view of a handle of the inner member of FIGS. 10A and 10B.

FIG. 14 shows a portion of a uterine measurement device outer sheath.

Like reference symbols in the various drawings indicate like elements.

### DETAILED DESCRIPTION

A uterine length measurement device is provided for directly measuring the length of a uterus. A first elongate member having a distal end, a proximal end and a lumen, is configured for insertion into an endocervical canal. The distal end is configured for insertion to approximately an internal cervical os of the endocervical canal. A second elongate member having a distal end and a proximal end is configured to move within the lumen of the first elongate member. The distal end can protrude from the distal end of the first elongate member and is configured for insertion to approximately the fundus of a uterus. Positioning the distal end of the first elongate member at approximately the internal cervical os and moving the second elongate member relative to the first elongate member to position the distal end of the second elongate member at approximately the fundus of the uterus provides a direct measurement of a length of the uterus.

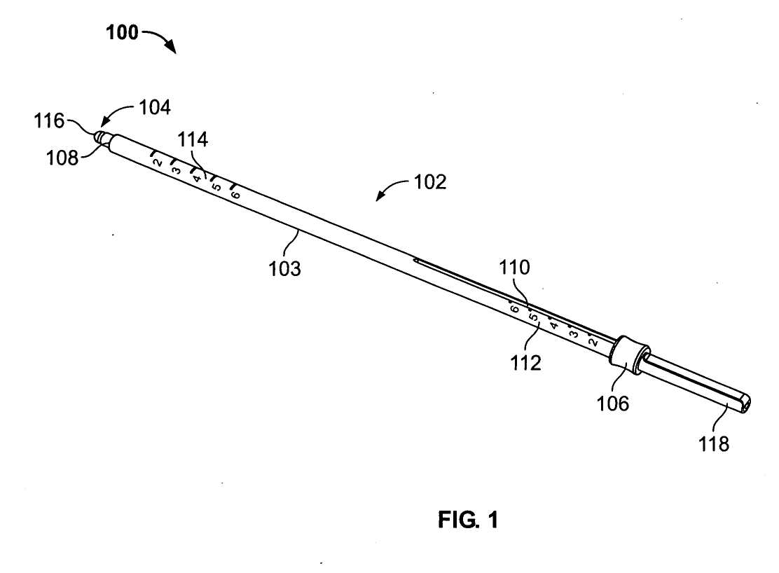

FIG. 1 shows an implementation of a uterine measurement device 100. The uterine measurement device 100 includes an outer sheath 102, an inner member 104, and a control knob 106. The outer sheath 102 includes an elongate lumen 103 having a proximal and distal end. The outer sheath 102 is configured to facilitate insertion into the endocervical canal.

The outer sheath 102 can include a reduced diameter step portion 108 at the distal end. The step portion 108 has an outer diameter less than an outer diameter of the outer sheath 102, and is configured for insertion to approximately an internal cervical os of the endocervical canal. The step portion 108 provides tactile feedback to a user for identifying the location of the internal cervical os of the endocervical canal. For example, the step portion 108 can be configured such that the outer sheath 102 cannot advance beyond the internal cervical os.

Specifically, a user can facilitate the identification of the internal cervical os using the step portion 108 through a dilation process. One or more dilators can be used to achieve a desired cervical opening. Due to the elastic differences between the inner cervical os and the cervical canal, a change in resistance (or constriction of the inner cervical os) can be identified according to a small difference in diameter between the step portion 108 and the outer sheath 102.

For example, if the outer sheath 102 has an 8 mm diameter with the step portion 108 having a 6 mm diameter, the user would dilate the cervix to 6 mm. As the physician advanced the uterine measurement device 100 into the cervical canal the step portion 108 would pass with much less resistance through the inner cervical os. The outer sheath 102 diameter of 8.0 mm would provide substantially more resistance and the physician would be able to feel this resistance and associate it with the location of the internal os.

The uterine measurement device 100 includes a handle 118 attached to the proximal end of the outer sheath 102. In one implementation, the handle 118 is an extension of the outer sheath 102. In another implementation, the handle 118 is a separate component coupled to the outer sheath 102. The handle 118 can be configured for user manipulation including finger grips or other tactile features allowing the user to hold the uterine measurement device 100.

In the implementation shown, the outer sheath 102 includes a first set of graduations 112 positioned near the proximal end. The first set of graduations 112 can provide a set of unit graduations configured to provide a length measurement of a uterine cavity. The outer sheath 102 can optionally include a second set of graduations 114 positioned near the distal end. The second set of graduations 114 can provide a set of unit graduations configured to provide a length measurement of an endocervical canal. The unit graduations on each set of graduations 112 and 114 can demarcate unit measurements, for example, in centimeters, millimeters, or some other unit.

The outer sheath 102 includes a slot 110 extending longitudinally in the proximal region. The slot can extend radially through one side of a wall of the outer sheath 102 from an outer radius to an inner radius, or can extend through both walls of the outer sheath 102, e.g., along a diameter of the outer sheath 102. The slot 110 allows a control knob 106 to couple to the inner member 104, such that by moving the control knob 106 along the length the slot, the inner member 104 is guided within the outer sheath 103 and can move between a retracted and an extended position. In one implementation, the slot 110 extends substantially the length of the first set of graduations 112.

In the uterine measurement device 100 shown in FIG. 1, the outer sheath 102 is configured for insertion into the endocervical canal. The outer sheath 102 can be substantially rigid in a compressive direction axially with respect to the distal and proximal ends as well as non-axially. The outer sheath 102 can be rigid axially such that a user is provided a tactile sensation when the internal cervical os is engaged by the step portion 108.

The inner member 104 is movable and has a proximal and a distal end and is configured to move within the lumen 103 provided by the outer sheath 102. The inner member 104 includes a tip 116 at the distal end. The distal end of the inner member 104 extends from the distal end of the outer sheath 102, such that the tip 116 can be advanced to approximately the fundus of the uterus. In one implementation only the tip 116, of the inner member 104, protrudes from the outer sheath 102 when the inner member 104 is in a retracted position. The tip 116 can be configured to be atraumatic to reduce a risk of injury when contacting uterine tissue (e.g., to reduce a risk of perforating the uterine wall). For example, as shown in FIG. 1, the tip 116 has a rounded surface that distributes the pressure generated by contact between the tip 116 and uterine tissue over a larger surface area, reducing the risk of damage. The inner member 104 can be flexible to allow a degree of bending necessary to locate the fundus of a curved uterus.

The control knob 106 allows the user to control movement of the inner member 104 relative to the outer sheath 102. In one implementation, the control knob is fixedly attached to the inner member 104, such that a movement of the control knob 106 provides a corresponding movement of the inner member 104. For example, if the control knob 106 is moved (e.g., through user manipulation) toward the distal end of the outer sheath 102, the inner member 104 extends from the distal end of the outer sheath 102. The control knob 106 can move along the outside of the outer sheath 102. For example, the control knob 106 can include a ring shape surrounding the outer sheath 102 with a connector (e.g., pin connector) to the inner member 104 extending through the slot 110. The slot 110 can thereby function as a guide, defining the range over which the control knob 106 and the inner member 104 can move. The control knob 106 can be configured to facilitate user manipulation, for example, including finger grips or other tactile features allowing the user to control the movement of the control knob.

In one implementation, the control knob 106 can lock the inner member 104 of uterine measurement device 100 in a retracted position. For example, a notch can be included orthogonal to the end point of the slot 110 at the proximal end of the uterine measurement device 100 such that a rotation of the control knob 106 in a direction of the notch can lock the inner member 104 and a reverse rotation from the locked position can unlock the inner member 104.

In another implementation, the inner member 104 can be threaded within the outer sheath 102, and a control knob can be rotated by a user to thread the inner member 104 into an extended or retracted position. In this implementation, the control knob includes an inner thread that mates with a thread formed on the exterior of the inner member 104, and rotating the control knob translates the inner member 104 axially. Other configurations can be used to translate the inner member 104 within the outer sheath 102 to move between the extended and retracted positions, and the techniques described herein are merely exemplary.

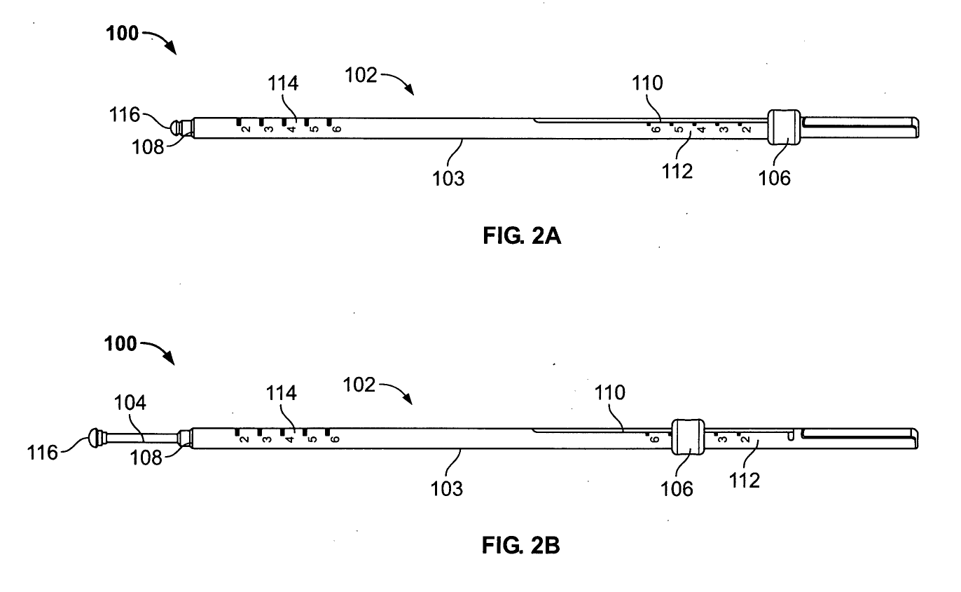

FIGS. 2A and 2B illustrate the uterine measurement device 100 in the retracted and extended positions, respectively. In FIG. 2A, the uterine measurement device 100 is shown in the retracted position. In the retracted position, the control knob 106 is positioned toward the proximal end of the slot 110 in the outer sheath 102. The inner member 104 is contained within the lumen provided by the outer sheath 102 such that only the tip 116 of the inner member 104 protrudes from the proximal end of the outer sheath 102.

In FIG. 2B, the uterine measurement device 100 is shown in the extended position. In the extended position, the control knob 106 is moved from the proximal end of the slot 110 toward the distal end of the slot 110 formed in the outer sheath 102. Consequently, as shown in FIG. 2B, the inner member 104 is shown extended from the outer sheath 102. In one implementation, a position of the control knob 106 relative to the first set of graduations 112 provides a measurement of the extended distance of the inner member 104, which, in use, can correlate to the length of the uterine cavity.

In one implementation, the uterine measurement device 100 can be disposable. The outer sheath 102 can be formed from injection molded thermoplastic, metal, or other material. In one implementation, the outer sheath 102 can be formed from plastics such as ABS, polystyrene, Peek, polycarbonate, or Ultem. In one implementation, the outer sheath 102 can be formed by injection molding two longitudinal halves, which are then attached together, for example, through the use of an adhesive or other bonding technique. Alternatively, the outer sheath 102 can be machined from a solid rod or tube of material.

The inner member 104 can be composed of injection molded thermoplastic. The plastic material can include polystyrene, LDPE, HDPE, a blend of LDPE/HDPE, polycarbonate, ABS, Peek, Delrin, or other suitable materials. The tip 116 and the shaft of the inner member 104 can be assembled from separate components or molded as a single component. The inner member 104 can be formed to include a curvature suitable for easing passage of the inner member 104 through the uterus. The curvature of the inner member 104 can be configured in any number of shapes and degrees of curvature, including, for example, an average curvature of a uterus.

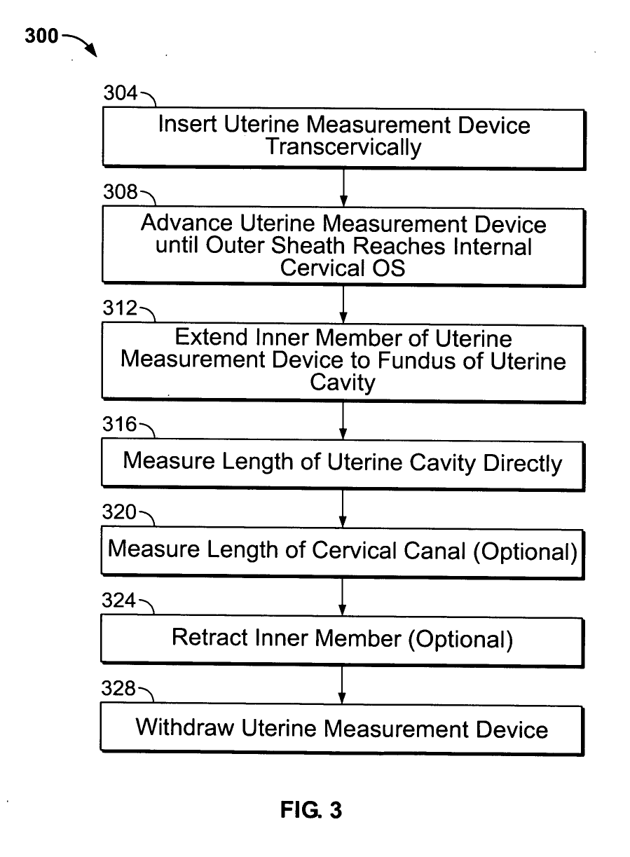

FIG. 3 shows a process 300 for using a uterine measurement device to directly measure the uterine cavity length. For illustrative purposes, the process 300 shall be described in reference to the implementation of the uterine measurement device shown in FIGS. 1, 2A and 2B, however, it shall be understood that the process 300 can be carried out using other implementations of the uterine measurement device.

A user, such as a physician or other medical professional, transcervically inserts the uterine measurement device (step 304). The uterine measurement device can be inserted in the retracted position with the inner member 104 within the lumen of the outer sheath 102. In one implementation, the user can first dilate the cervix to a diameter less than or equal to the reduced diameter of the step portion 108 of the outer sheath.

The user advances the uterine measurement device until the step portion 108 of the outer sheath reaches the internal cervical os (step 308). For example, the user can receive tactile feedback when the outer sheath 102 reaches the internal cervical os. The step portion 108 can be configured to provide the user with resistance indicating that the internal cervical os has been reached, as discussed above. Additional structures can also be added to the outer sheath 102 at substantially the step portion 108 in order to provide greater resistance upon reaching the internal cervical os. For example, FIG. 14 shows the addition of small wings 1402 to the distal portion of the outer sheath 102 in order to increase the tactile resistance when the outer sheath 102 reaches the internal cervical os. Other structures can be used to provide a feature at the distal end of the outer sheath 102 to provide tactile feedback indicating the position of the internal cervical os. For example, the distal end of the outer sheath 102 can include a balloon structure or raised groove, which increase the tactile resistance at the internal cervical os.

After inserting the outer sheath 102 of the uterine measurement device 100 to approximately the internal cervical os, the user extends the tip 116 of the inner member 104 of the uterine measurement device to approximately the fundus of the uterine cavity (step 312). The user can extend the inner member 104 by manually moving the control knob 106 coupled to the inner member 104 through the outer sheath 102. For example, the user can advance the control knob 106 along the length of the outer sheath 102 toward the distal end in order to extend the inner member beyond the distal end of the outer sheath 102 by a corresponding amount. The user locates the fundus of the uterine cavity by tactile feel of axial resistance from the inner member 104 once the tip 116 of the inner member contacts the uterine wall at the fundus.

Once the fundus is located, the user can directly measure the length of the uterine cavity (step 316). The distance the control knob 106 traverse when moving from the retracted position to the extended position correlates to the measurement of the length of the uterine cavity. As discussed above, in the implementation shown in FIGS. 1-2B, the outer sheath 102 include a first set of graduations 112 that indicate different measurement amounts. The position of the control knob 106 relative to the graduations provides a direct measurement length for the uterine cavity.

Additionally, the user can optionally directly measure the length of the endocervical canal (step 320). The user can measure the length of the endocervical canal according to a second set of graduations 114 positioned near the distal end of the outer sheath 102. The length of the endocervical canal is measured from the internal cervical os to the external orifice. In one implementation, the user can move a collar along the outer sheath 102 until the external orifice is reached. The position of the collar relative to the second set of graduations 114 provides an indication of the length of the endocervical canal.

After measuring the uterine cavity length, the user can then retract the inner member 104 back within the outer sheath 102 for extraction of the uterine measurement device 100 from the patient (step 324). Alternatively, the user can leave the inner member in the extended position, for example, to read the length measurement at a later time. In one implementation, the control knob 106 can be locked into position, with the inner member extended, such that the measurement position is maintained for later review. The uterine measurement device 100 can then be withdrawn transcervically (step 328).

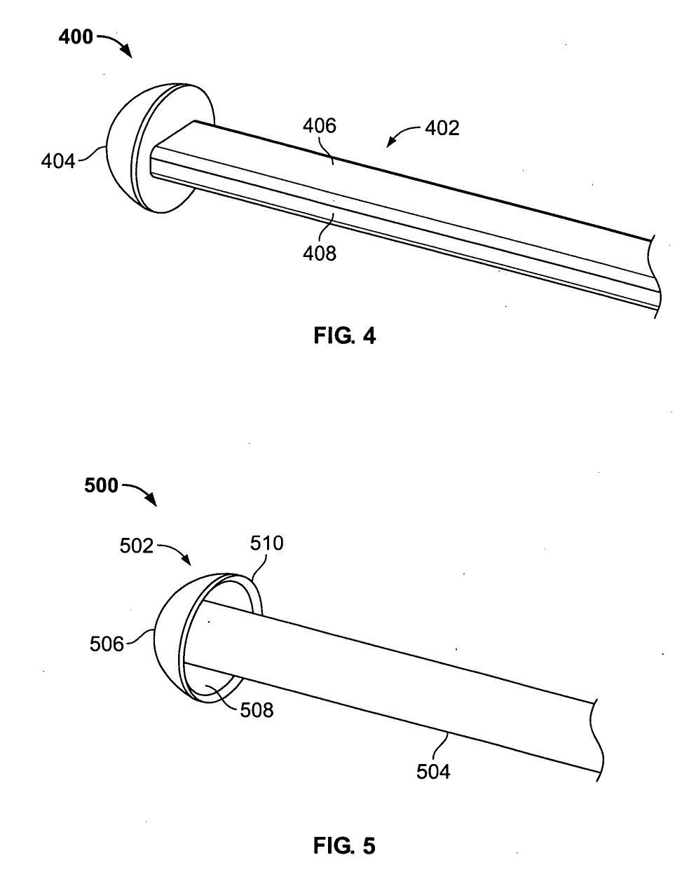

FIG. 4 shows a detailed view of a distal portion of one implementation of an inner member 400. The inner member 400 includes a shaft 402 (partially shown) and a tip 404. The shaft 402 has a ribbon shape having a rectangular cross section with a width 406 and a height 408. The rectangular cross section of the shaft 402 provides a preferential bending plane for the inner member 404. The width 406 is greater than the height 408, such that the inner member 404 has a greater flexibility along a plane including the width 406 then along a plane including the height 408. The inner member 404 can be configured to provide the preferential bending along the plane of the triangular uterine cavity, which can be curved upwards or downwards out of the plane. The flexible inner member therefore can flex in order to accurately locate the fundus of the uterine cavity when the uterus is curved upwards or downwards. Additionally, the lesser flexibility provided in the plane including the height 408 reduces the chance of bending the inner member 400 such that the tip 404 enters either of the fallopian tubes.

In an alternative implementation, the inner member can be formed from one or a combination of materials in order to provide variable flexibility along the length of the inner member. The variable flexibility of the inner member can be configured to provide a greater degree of flexibility along the distal end of the inner member and a lesser degree of flexibility at the proximal end. In one implementation, the degree of flexibility of the inner member can incrementally increase from the proximal end to the distal end. In one implementation, the variable flexibility can be provided geometrically. For example, the shaft of the inner member can taper from the proximal end to the distal end in order to provide greater flexibility at the distal end.

FIG. 5 shows one implementation of a tip 502 of an inner member 500. The tip 502 is attached to the distal end of a shaft 504 (partially shown) of the inner member 500. The tip 502 is configured in a cup shape having a convex shaped outer surface 506 and a concave inner surface 508. An edge 510 demarcates the rim of the cup separating the outer surface 506 and the inner surface 508. The convex outer surface 506 is configured to provide an atraumatic surface for contacting the uterine wall. The concave inner surface 508 is configured to collect endometrial tissue from the uterine wall as the edge 510 scrapes along the uterine cavity when the inner member 500 is retracted. The concave inner surface 508 collects the tissue scrapings for testing or other purposes by a user or other individual such as a lab technician.

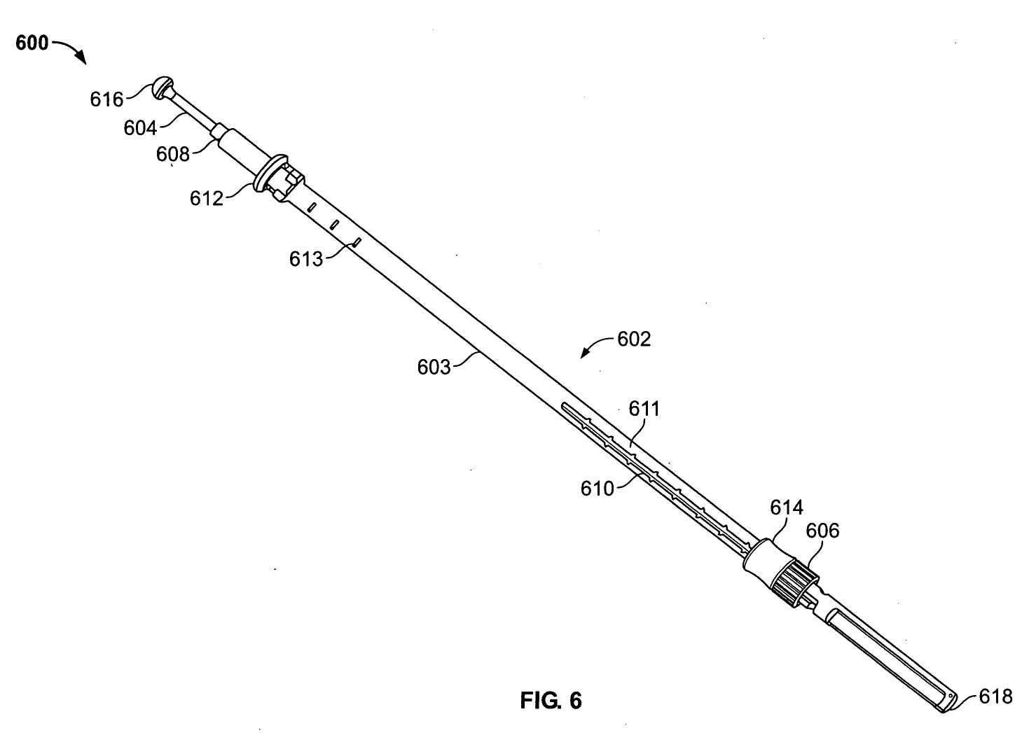

FIG. 6 shows another implementation of a uterine measurement device 600. The uterine measurement device 600 is similar to the uterine measurement device 100 shown in FIG. 1, and also includes an outer sheath 602, inner member 604, and a control knob 606. The outer sheath 602 includes an elongate lumen 603 having a proximal and distal end and a step portion 608 at the distal end of the outer sheath 602.

The uterine measurement device 600 also includes a handle 618 attached to the proximal end of the outer sheath 602. The outer sheath 602 includes a first set of graduations 611 along the proximal end. The first set of graduations 611 can provide a set of unit graduations configured to provide a length measurement of a uterine cavity. The outer sheath 602 can optionally include a second set of graduations 613 along the distal end. The second set of graduations 613 can provide a set of unit graduations configured to provide a length measurement of an endocervical canal.

The outer sheath 602 also includes a slot 610 along the proximal end. The slot can extend radially through a single wall of the lumen formed by the outer sheath 603 from the outer diameter to the inner diameter, or through both walls of the lumen 603, e.g., along a diameter of the lumen. The slot 610 allows the control knob 606 to attach to the inner member 604.

The uterine measurement device 600 includes the following feature that is not included in the device 100 shown in FIG. 1. A movable element is coupled to the outer sheath 602 for measuring the length of the endocervical canal according to the second set of graduations 613. In the implementation shown, the element is a collar 612. However, other configurations of the movable element are possible. During a measurement operation, the user can manually move the collar 612 along the outer sheath 602 toward the distal end until the external os of the cervix is reached. In one implementation, the collar 612 is configured as a ring that can slide along the outer surface of the outer sheath 602. After removing the uterine measurement device 600 from the patient, the user can view a direct measurement of the endocervical canal length according to the position of the collar 612 relative to the second set of graduations 613.

The uterine measurement device 600 also can include the following additional feature. The control knob 606 can be lockable, allowing the user to control movement of the inner member 604 relative to the outer sheath 602. In one implementation, the control knob is fixedly attached to the inner member such that a movement of the control knob 606 provides a corresponding movement of the inner member 604. For example, if the control knob 606 is moved (e.g., through user manipulation) toward the distal end of the outer sheath 602, the inner member 104 extends from the distal end of the outer sheath 602. The control knob 606 can move along the outside of the outer sheath 602. For example, the control knob 606 can include a ring shape surrounding the outer sheath 602. The control knob 606 can be attached to the inner member 604 through the slot 610 using, for example, a pin connector.

Additionally, the lockable control knob 606 includes a locking collar 614 configured to lock the control knob 606 in place along the outer sheath 602. The locking collar 614 allows the user to lock the control knob at any position within the movable range of the control knob 606 along the outer sheath 602. For example, the user can lock the control knob 606 once the fundus has been located such that the uterine measurement device 600 can be withdrawn and the uterine length recorded later according to the locked position of the control knob 606. In one implementation, the locking collar 614 is configured to tighten around the outer sheath 602 to lock the control knob 606. For example, the locking collar 614 can be a rotatable collar positioned at the proximal end of the control knob 602. Rotation of the locking collar 614 tightens the locking collar 614 around the outer sheath 602 providing a friction hold of the control knob 602. Rotation of the locking collar 614 in the opposite direction can then untighten the locking collar 614, releasing the control knob 602. Other locking mechanisms can be used, for example, a pin vise clamp, threaded collar or other structure.

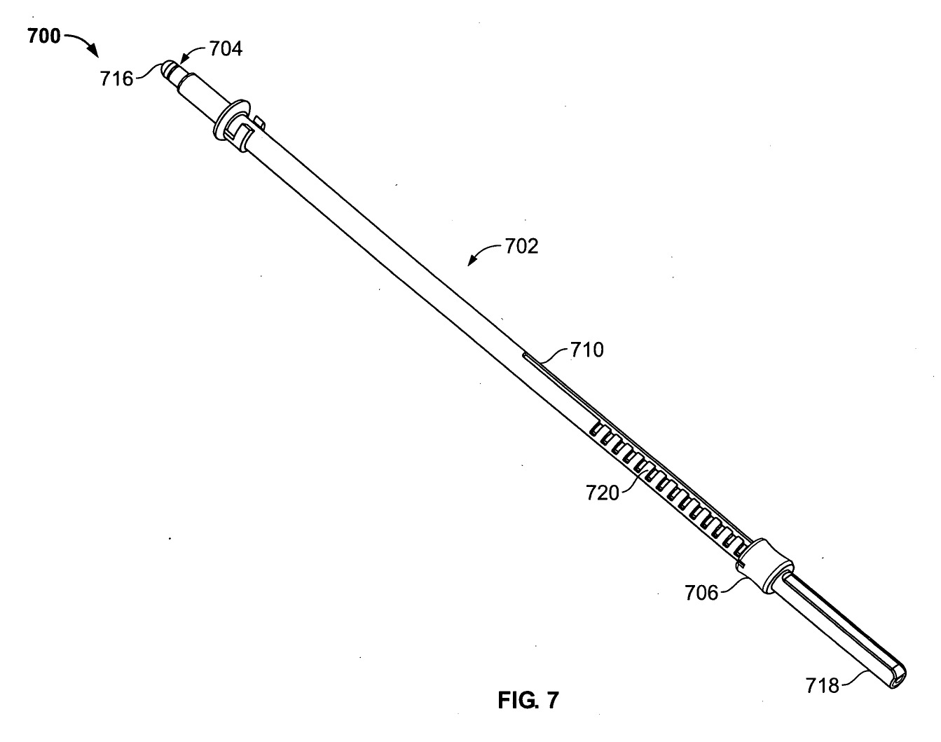

FIG. 7 shows another implementation of a uterine measurement device 700. The uterine measurement device 700 includes an outer sheath 702, inner member 704, tip 716, handle 718, and control knob 706. The outer sheath 702 includes a slot 710 and a series of locking grooves 720. The slot 710 runs along a portion of the axis of the outer sheath 702 and provides for coupling the control knob 706 to the inner member 704. The length of the slot 710, along the axis of the outer sheath 702, provides a range for extending or retracting the inner member 704 from the distal end of the outer sheath 702.

The locking grooves 720 are formed in the surface of the outer sheath 702 adjacent and orthogonal to the slot 710. The locking grooves can be provided in measured intervals along the length of the slot 710. In one implementation, each locking groove 720 is separated by substantially one half a centimeter. Other groove separations are possible and can be either uniform or non-uniform. The control knob 706 can be configured to engage a locking groove 720, for example, by rotating the control knob 706 in the direction of a locking groove 720. In operation, for example, once the user has extended the inner member 704 to the fundus, the user can engage the nearest locking groove 720 to lock the control knob 706. The uterine measurement device 700 can then be removed and the uterine length later recorded based on the position of the locked control knob 706.

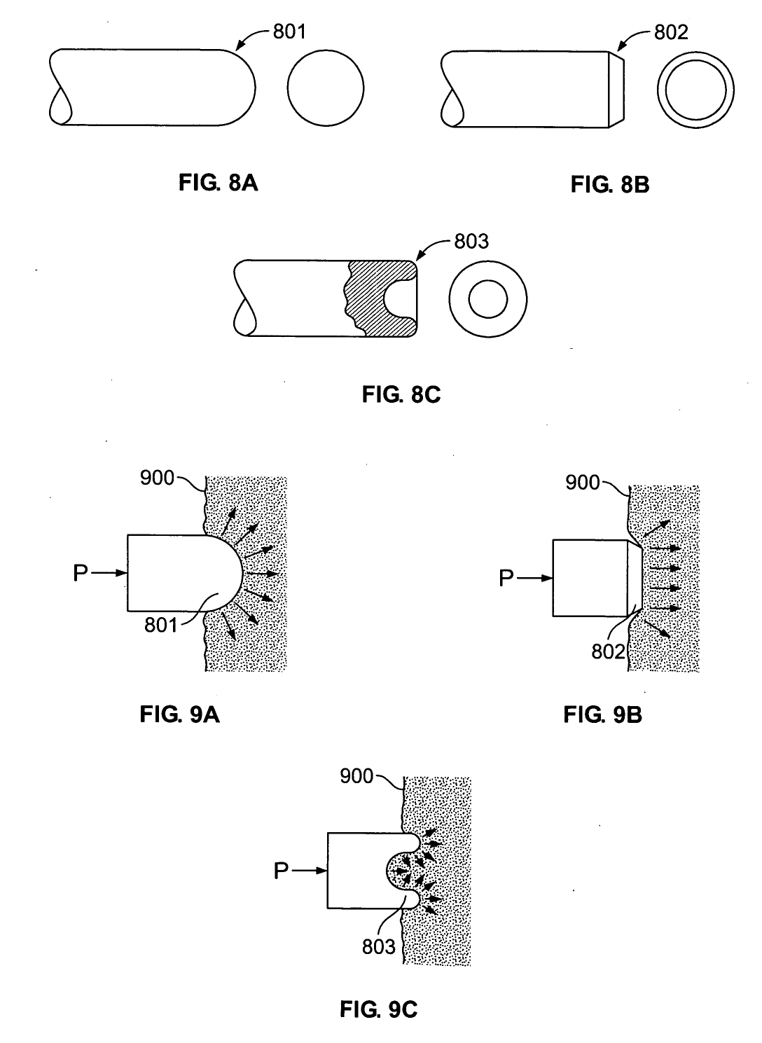

Referring again to FIG. 1, as mentioned above, the uterine measurement device can be configured with differently shaped tips 112. Referring now to FIGS. 8A-C and 9A-C, three implementations of a tip 801, 802 and 803 are shown. The distal tips 801-803 include atraumatic geometry configured to resist perforation of the uterine wall 900 by reducing stress on the uterine wall 900. The examples of atraumatic geometry that are shown in FIGS. 8A-C include a full radius tip 801, a chamfered tip 802 and a concave tip 803 respectively.

As shown in FIGS. 9A-C, different atraumatic distal tip geometries produce different axial loads P on the uterine wall 900. FIG. 9A illustrates the forces on the uterine wall 900 (shown as arrows) by a distal tip 801 configured as a full radius tip. FIGS. 9B and 9C similarly illustrate the forces on the uterine wall 900 by distal tips configured as a chamfered tip 802 and a concave tip 803 respectively. A full radius tip 801 as shown in FIG. 9A, resists scraping the uterine wall 900 during insertion into the uterus, but can tend to divide tissue when an axial load is applied. A chamfered tip 802, as shown in FIG. 9B, resists scraping the uterine wall 900 moderately well and better resists puncturing the wall 900 relative to a full radius tip 801. A chamfered tip 802 tends to create less radial force (indicated by arrows) in tissue, in comparison to a full radius tip 801 as shown in FIGS. 9A and 9B. Concave tip 803 can significantly protect against scraping and puncturing the uterine wall 900 and tends not to divide tissue. As shown in FIG. 9C, although the concave tip 803 does generate some radial forces (indicated by arrows) that develop tensile hoop stress on the outer perimeter, the hoop stress produced in the central region is compressive (indicated by arrows).

In an alternative implementation, a uterine measurement device can be provided that includes an inner member having an end cap at the distal end that can have an open position and a closed position. The end cap can be in the closed position during insertion into the uterus. Under conditions where there is a risk of the uterine measurement device perforating the uterine wall, the end cap automatically switches to the open position. The open position provides an enlarged surface area of the distal end of the inner member of the uterine measurement device that is in contact with the uterine wall and resists perforation of the uterine tissue.

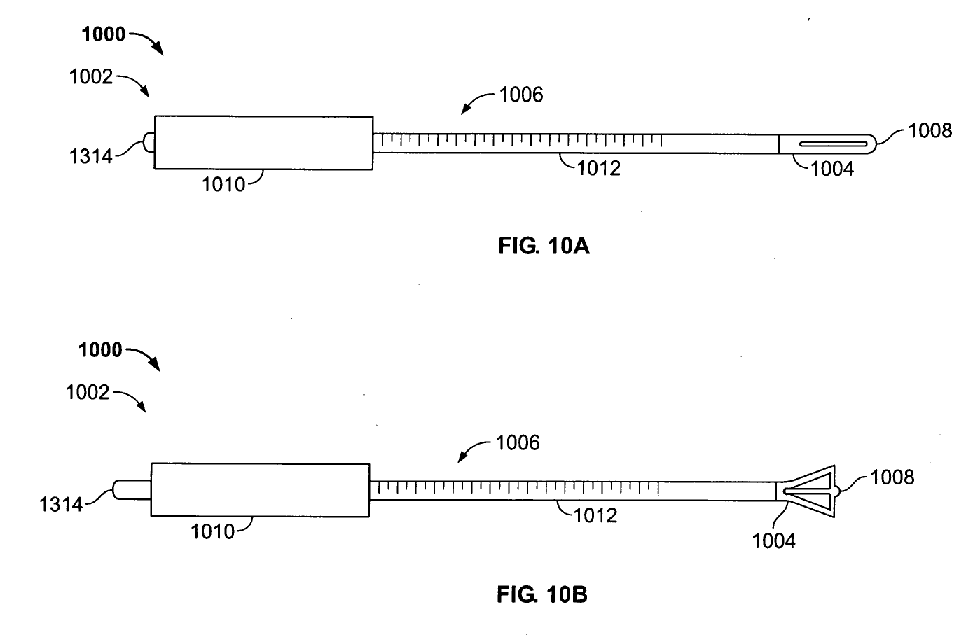

Referring to FIGS. 10A and 10B, one embodiment of an inner member 1002 of a uterine measurement device is shown. The inner member 1002 can be incorporated into a uterine measurement device, such as the device 100 shown in FIG. 1, in which case, the inner member 1002 would replace the inner member 104 shown in FIG. 1. The inner member 1002 has an open and a closed position. In FIG. 10A the inner member 1002 is in a closed position, and is configured to facilitate insertion into a uterus. In FIG. 10B the inner member 1002 is in an open position; the end cap 1004 of the inner member 1002 has changed geometry from having a relatively small distal tip to having an enlarged surface area.

In the embodiment depicted, the inner member 1002 includes an elongate member 1006 having distal and proximal ends. The elongate member 1006 is generally rigid axially yet flexible and/or malleable non-axially. As such, the elongate member 1006 is rigid in the compressive direction with respect to the elongate member's distal and proximal ends, and flexible out of a longitudinal axis of the elongate member 1006. The elongate member 1006 can be rigid in the compressive direction such that a user is provided a tactile sensation when the fundus of the uterus is engaged.

As shown in FIGS. 10A and 10B, the end cap 1004 is connected to the distal end of the elongate member 1006. The end cap 1004 can be configured in a closed position for when the elongate member 1006 is inserted into the uterus and when sounding the uterus under normal conditions (see FIG. 10A). Additionally, the end cap 1004 is in the closed position when partially or wholly within the outer sheath (e.g., outer sheath 102 in FIG. 1) of the uterine measurement device 1000. The end cap 1004 can further be configured to automatically switch into an open position of enlarged surface area when a force is applied to a distal tip 1008 of the end cap 1004 by the uterine tissue in excess of a threshold force (see FIG. 10B). That is, the surface area of the end cap 1004 projected onto a plane substantially perpendicular to a longitudinal axis of the elongate member 1006 is enlarged in the open position. In the open position the enlarged geometry of the end cap 1004 resists penetration of the uterus by the inner member 1002. The inner member 1002 can also include a handle 1010 connected to the proximal end of the elongate member 1006. The handle 1010 can replace or be integrated with the handle 118 coupled to the outer sheath 102 of the uterine measurement device 100 shown in FIG. 1.

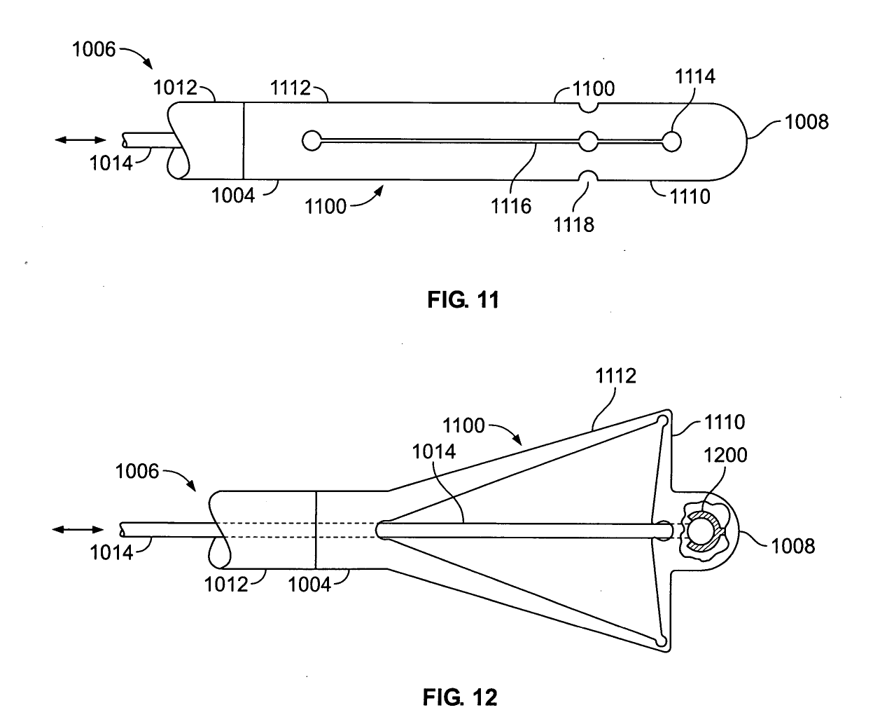

Referring also to FIG. 11, in the embodiment depicted, the elongate member 1006 includes a shaft 1012 and a rod 1014 disposed within the shaft 1012. The rod 1014 spans the length of the elongate member 1006 and is attached to the distal end of the end cap 1004. Referring to FIG. 12, in one embodiment the rod 1014 is attached to the distal tip 1008 of the end cap 1004 by a snap fit 1200 connection. The snap fit 1200 can be in the form of a clevis-type coupling (see FIG. 12) a threaded feature, a pin, a bonding agent or any other suitable means. Where the snap fit 1200 is a clevis snap fit, a rotational degree of freedom can be provided between the rod 1014 and the distal tip 1008 of the end cap 1004.

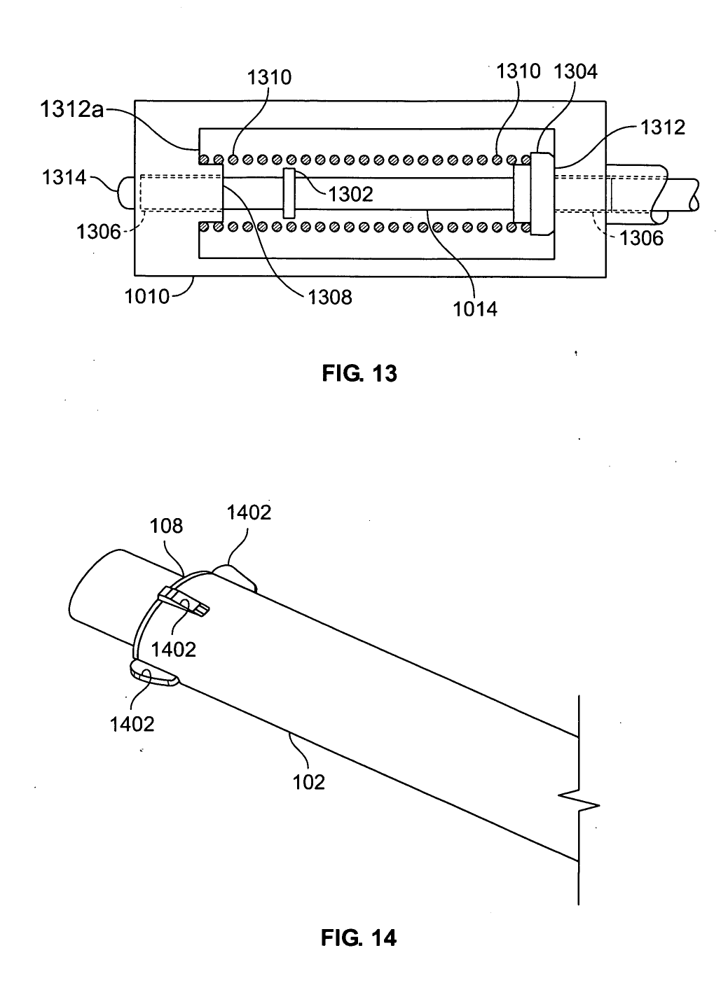

Referring to FIG. 13, a cross-sectional view of the handle 1010 is shown. The rod 1014 can include a hardstop 1302 attached to the rod 1014 for limiting translational movement of the rod within the handle 1010. Also shown in FIG. 13, a retainer 1304 can be attached to the rod 1014 within the handle 1010, which is described further below.

Referring to FIGS. 11 and 12, the end cap 1004 can include one or more deployable fins 200 that provide a convertible arrangement for the end cap 1004 between a closed position (see FIG. 11) and an open position (see FIG. 12). The open position provides an enlarged surface area at the distal end of the inner member 1002. Deployment of the end cap 1004 to the open position is triggered when a force exceeding a threshold force is exerted on the distal tip 1008 of the end cap 1004 and transmitted down the shaft 1012. That is, when the inner member 1002 reaches the end of the uterus, or another portion of uterine wall, and a user continues pushing on the proximal end of the inner member 1002, if the resisting force exerted by the uterine wall on the end cap 1004 exceeds the threshold force, then the open position is triggered.

As shown in FIG. 12, in one embodiment, when the open position is triggered, two fins 1100 deploy radially outwardly to provide an enlarged surface area. The fins 1100 can be formed from shorter links 1110 and longer links 1112. The length of the shorter links 1110 relative to the longer links 1112 can follow an approximate 1:3 ratio. Additionally, where the deployed shorter links 1110 are substantially perpendicular to the long axis of the inner member 1002, the longer links 1112 are disposed at an angle including but not limited to, for example 25-30 degrees. In one embodiment, the shorter links 1110 are approximately 0.25 to 1 centimeter in length, while the longer links 1112 are approximately 0.75 to 3 centimeters in length. In another embodiment, the shorter links 1110 are approximately 0.7 centimeters in length and the longer links 1112 are approximately 2.1 centimeters in length. The outward deployment of the shorter links 1110 can include rotation of the shorter links 1110 through a larger angle than that rotated through by the connected longer links 1112. Particularly, the shorter links 1110 can be configured to deploy substantially 90 degrees to the long axis of the elongate member 1006, while the longer links 1112 deploy substantially 30 degrees to the long axis of the elongate member 1006 (see FIG. 12). The deployed shorter links 1110 and longer links 1112 create a substantially rigid, stable triangular configuration capable of withstanding substantial loads without buckling.

The shorter links 1110 and longer links 1112 of the fins 1100 can be injection molded links, pinned rigid links, resilient wire or other suitable formed links. When the end cap fins 1100 are injection molded, the end cap 1004 can have one or more slots 1116 defining fin 1100 width and one or more holes 1114 in the slot 1116. The holes 1114 are configured to define shorter link 1110 and longer link 1112 length, and provide an area of increased bending stress, thereby providing a “living hinge” at the ends of the fins 1100. A living hinge can be, for example, a molded thin flexible bridge of material (e.g., polypropylene or polyethylene) that joins two substantially rigid bodies together. Additional one or more holes 1118 in the end cap 1004 located adjacent to the one or more slots 1116, can be configured to enhance the living hinge separating the shorter links 1110 and longer links 1112.

The inner member 1002 includes a feature to sense when to switch from a closed to an open position, and a feature to deploy into the open position. In the embodiment shown, a mechanical deployment mechanism both senses when a threshold force is exceeded and automatically deploys the fins 1100 into the open position. Referring again to FIGS. 10 and 13, the deployment mechanism can be a mechanical assembly, housed within the handle 1010. The handle 1010 is attached to the elongate member 1006 at or substantially near to the proximal end. Other deployment mechanisms for converting from the closed position to the open position can be used, including electrical means by incorporating a force sensitive resistor (FSR) at the distal tip 1008. When the force exerted against the FSR exceeds a threshold value, the resistance of the FSR changes from one state to a different state. A detector located, for instance, in the handle 1010 can detect the change and trigger the release of a braking means holding the rod 1014 in place, allowing the end cap 1004 to deploy. Still another embodiment could employ a pneumatic means, whereby the force applied at the distal tip translates through the rod 1014, which could in turn bear on a plunger in a reservoir inside handle 1010. When the pressure inside the reservoir reaches the threshold value, a pressure releasing means could trigger the end cap 102 to change to its deployed condition.

An orientation indicator can be provided to indicate to a user the proper orientation of the inner member 1002 relative to the uterus. For example, where the fins 1100 of the inner member 1002 deploy in a plane, the proper orientation substantially aligns the plane with the plane of the substantially flat uterus to ensure safe deployment of the fins 1100. The orientation indicator can be positioned substantially near the proximal end of the inner member 1002. The orientation indicator can be a marking on the surface, or a tactile indicator at the proximal end of the inner member 1002. In one embodiment, the proximal end of the handle 1010 can include an orientation indicator in the form of a flattened planar side that coincides with the plane of deployment of the fins 1100. In one embodiment, the plane of handle 1010 itself can indicate the plane of deployment of the fins 1100. Additionally, the orientation indicator can be positioned on the outer sheath of the uterine measurement device 1000 (e.g., outer sheath 102 of FIG. 1) or on the control knob (e.g., control knob 106 of FIG. 1)

In the embodiment shown in FIG. 13, the mechanical assembly included within the handle 1010 includes journals 1306 for providing a single translational degree of freedom to the rod 1014, and a boss 1308 for contacting the hardstop 1302 of the rod 1014, thereby limiting the translational movement of the rod 1014. The mechanical assembly further includes a means to govern the threshold force required to trigger conversion to the open position, e.g., to deploy the fins 1100. In the embodiment depicted, the means for governing the threshold force include a spring 1310, e.g., a compression spring. The spring 1310 can be preloaded between the handle wall 1312a at the handle's proximal end and the retainer 1304 connected to the rod 1014 near the handle wall 1312b at the handle's distal end. The retainer 1304 is constrained by the adjacent handle wall 1312b to maintain the spring 1310 preload. Alternatively, the governing means can include a pressurized gas in a cylinder formed within handle 1010, wherein retainer 1304 can be configured as a piston capable of translating through the cylinder.

When a uterine measurement device incorporating an inner member 1002 as shown in FIGS. 10A-B is inserted into a uterus, and the distal tip 1008 of the end cap 1004 presses against a uterine wall, a resistance force exerted by the uterine wall 600 (see FIG. 8A-C) on the distal tip 1008 is transmitted along the rod 1014 to the retainer 1304. Typically, measurement of the uterus length presents little risk of perforation using the uterine measurement device, since the end of the uterus can be identified by tactile sensation without exceeding the threshold force.

Under certain circumstances, e.g., through inadvertence, accident, anatomical divergence or stenosis of the uterus, the measuring process can result in forces on the uterine wall 600 that could perforate the uterus with the uterine measurement device. Once a force approaching, but substantially lower than a force capable of perforating the uterine wall 600, i.e., the threshold force, is transmitted to the retainer 1304, the force preloaded in the spring 1310, i.e., the threshold force, begins to compress the spring 1310. As the spring 1310 compresses, the retainer 1304 moves away from the adjacent handle wall 1312b and translates the rod 1014 through the journals 1306. The rod's translation is limited by the hardstop 1302 contacting the boss 1308. The translation of the rod 1014 relative to the shaft 1012 draws the distal tip 1008 of the end cap 1004 toward the handle 1010, thereby deploying the fins 1100 (see FIG. 12) and creating the desired enlarged surface area for resisting penetration of the end cap 1004 into the uterine wall 600.

After deployment, the fins 1100 of the inner member 1004 can be returned to the undeployed state by e.g., physically pushing the proximal end of the rod 1014 to the undeployed position in the elongate member 1006, thereby returning the distal tip 500 of the end cap 1004 and accordingly the fins 1100 to their undeployed positions. Alternatively, in the embodiment depicted, once the force on the distal tip 1008 of the end cap 1004 is released, i.e., is less than the threshold force, the spring 1310 expands and automatically contracts the fins 1100. Once returned to the undeployed position, the uterine measurement device can safely be removed.

Referring again to FIGS. 10 and 13, the inner member 1002 can optionally include an indicator to indicate to a user of the uterine measurement device that the threshold force was exceeded and that the inner member 1002 has converted to the open position. In the embodiment depicted, the indicator is a protrusion 1314 from the handle 1010 that is continuously connected to the rod 1014. When the threshold force of the inner member 1002 is exceeded, translation of the rod 1014 causes the protrusion 1314 to further protrude from the handle 1010, thereby providing a signal or alert to the user. In other embodiments, the indicator can be both visual and audible and can be a mechanical or an electric device or a combination of the two. For example, where the indicator is the protrusion 1314, a colored section (e.g., yellow or red) can be revealed upon exceeding the threshold force when the indicator is caused to protrude further from the handle 1010 (not shown).

Alternative techniques can also be used to provide the measurement of the uterine cavity length. For example, electronic circuitry can be used. In one implementation, electrical contacts can be positioned at a predefined spacing along the outer sheath. The spacing interval can correspond to a desired measurement interval. Additionally, the interval can decrease as the distance from the proximal end of the outer sheath increases in order to provide increased measurement accuracy within a typical uterine cavity length range. Corresponding electrical contacts can be positioned on an interior surface of the control knob (e.g., positioned on the surface of the inner circumference of a ring shaped control knob). As the control knob moves along the outer sheath, the electrical contacts of the control knob mate with corresponding electrical contacts of the outer sheath in order to complete an electrical circuit. Logic associated with the various circuit pathways can determine the distance traveled along the outer sheath by the control knob according to which electrical contacts on the outer sheath were activated. The distance the control knob advanced is used to determine the uterine cavity length. In one implementation, a display, e.g., an LCD screen, can be used to provide a digital display to a user of the uterine cavity length.

In another implementation, the control knob can include an array of micro-switches positioned on the inner surface. Each micro switch can be configured to be switched on or off depending on whether the switch is in a raised or lowered position. The outer shaft can include an array of dimples along the outer shaft at predefined intervals. The interval can correspond to one or more desired measurement intervals. One or more of the micro switches can be toggled into the raised or lowered position at each measuring interval. In one implementation, the pattern of raised or lowered micro switches at a given measurement interval corresponds to a particular uterine length value. For example, for an array of 10 micro switches along the interior circumference of the control knob, at the first measuring interval (e.g., 1 cm), the outer shaft can have only one dimple such that only a single micro-switch is toggled, providing a signal corresponding to a length of 1 cm. At the next measuring interval (e.g., 1.5 cm), the outer sheath can have two dimples such that two micro-switches are toggled. Subsequent dimple patterns correspond to subsequent length measurement. In one implementation, the dimples at each interval are elongated to span between to the next measurement interval. The above examples are exemplary only; other electronic devices can be used to measure and/or display the uterine cavity length.

A number of embodiments of the invention have been described. Nevertheless, it will be understood that various modifications may be made without departing from the spirit and scope of the invention. Accordingly, other embodiments are within the scope of the following claims.

What is claimed is:

1. A uterine length measurement device, comprising:
a first elongate member having a distal end, a proximal end and a lumen, the elongate member configured for insertion into an endocervical canal where the distal end is configured for insertion to approximately an internal cervical os of the endocervical canal; and
a second elongate member having a distal end and a proximal end, the second elongate member configured to move within the lumen of the first elongate member, where the distal end can protrude from the distal end of the first elongate member and is configured for insertion to approximately the fundus of a uterine cavity,
wherein the device is configured such that positioning the distal end of the first elongate member at approximately the internal cervical os and moving the second elongate member relative to the first elongate member to position the distal end of the second elongate member at approximately the fundus of the uterus provides a direct measurement of a length of the uterine cavity.

2. The uterine length measurement device of claim 1, where the first elongate member further comprises:
a stepped portion at the distal end configured to facilitate locating the internal cervical os, the stepped portion having an outer diameter that is less than the outer diameter of the first elongate member.

3. The uterine length measurement device of claim 1, where the first elongate member further comprises:
graduations marked on at least a portion of a length of the first elongate member for indicating relative movement of the second elongate member to the first elongate member, the relative movement corresponding to the direct measurement of the length of the uterine cavity.

4. The uterine length measurement device of claim 3, further comprising:
a control knob located near the proximal end of the second elongate member; and
a slot formed in proximal region of the first elongate member and adjacent to the graduations and configured to receive the control knob;
where the control knob movable within the slot to advance and retract the second elongate member within the lumen of the first elongate member and the position of the control knob relative to the graduations when the distal end of the second elongate member is positioned at approximately the fundus of the uterus indicates the length of the uterine cavity.

5. The uterine length measurement device of claim 3, where the first elongate member further comprises:
second graduations marked on a second portion of the length of the first elongate member for measuring a length of the endocervical canal.

6. The uterine length measurement device of claim 5, further comprising:
a collar configured to move along the first elongate member to mark a position relative to the second graduations identifying a measurement of the endocervical canal.

7. The uterine length measurement device of claim 1, where the distal end of the second elongate member includes an atraumatic tip.

8. The length measurement device of claim 7, where the atraumatic tip further comprises a concave region configured to collect endometrial tissue when the atraumatic tip is positioned within the uterine cavity and retracted toward the proximal end of the first elongate member.

9. The uterine length measurement device of claim 1, where the second elongate member is substantially rigid compressively between the distal and proximal ends, and substantially flexible out of a plane of a longitudinal axis of the second elongate member.

10. The uterine length measurement device of claim 1, where the second elongate member is substantially flexible along a first plane out of a longitudinal axis of the second elongate member and substantially rigid along a second plane out of the longitudinal axis.

11. The uterine length measurement device of claim 1, where the flexibility of the second elongate member varies from the proximal to the distal end such that the distal end has a greater flexibility than the proximal end.

12. The uterine length measurement device of claim 1, where the second elongate member has a substantially rectangular cross section.

13. The uterine length measurement device of claim 1, where the second elongate member has a substantially circular cross section.

14. The uterine length measurement device of claim 1, where the second elongate member has a substantially oval shaped cross section.

15. The uterine length measurement device of claim 4, further comprising:
one or more locking grooves in the first elongate member configured to hold the control knob in place relative to the first elongate member when the control knob is rotated into a particular locking groove.

16. The uterine length measurement device of claim 4, further comprising:
a locking collar coupled to the control knob, the locking collar configured to tighten around the first elongate member to lock the control knob relative to the first elongate member.

17. The uterine length measurement device of claim 1, wherein the distal tip of the second elongate member is configured to be atraumatic.

18. The uterine length measurement device of claim 1, wherein the distal tip of the second elongate member is a full radius tip.

19. The uterine length measurement device of claim 1, wherein the distal tip of the second elongate member is a chamfered tip.

20. The uterine length measurement device of claim 1, wherein the distal tip of the second elongate member is a convex tip.

21. A uterine length measurement device, comprising:
a first elongate member having a distal end, a proximal end and a lumen, the elongate member configured for insertion into an endocervical canal where the distal end is configured for insertion to approximately an internal cervical os of the endocervical canal; and
a second elongate member having a longitudinal axis, a distal end and a proximal end, the second elongate member configured to move within the lumen of the first elongate member, where the distal end can protrude from the distal end of the first elongate member and is configured for insertion to approximately the fundus of a uterus; and
an end cap connected to the distal end of the second elongate member, the end cap being in a closed position when the second elongate member is inserted into the uterus and switching into an open position when a force applied to a distal tip of the end cap by the uterus exceeds a threshold force, where a surface area of the end cap projected onto a plane substantially perpendicular to the longitudinal axis of the second elongate member is enlarged in the open position as compared to in the closed position, and where the open position resists penetration of the end cap into a wall of the uterus;
wherein the device is configured such that positioning the distal end of the first elongate member at approximately the internal cervical os and moving the second elongate member relative to the first elongate member to position the distal end of the second elongate member at approximately the fundus of the uterus provides a direct measurement of a length of a uterine cavity.

22. The uterine length measurement device of claim 21, wherein the end cap includes one or more fin members, where the fin members are positioned along a longitudinal axis of the elongate member when the end cap is in the closed position and deploy laterally from the longitudinal axis when switching into the open position.

23. The uterine length measurement device of claim 21, further comprising:
a deployment mechanism, configured to switch the end cap from the closed position to the open position upon a force on the distal tip of the end cap exceeding the threshold force.

24. The uterine length measurement device of claim 23, wherein:
the end cap includes one or more fin members, where the fin members are positioned along a longitudinal axis of the elongate member when the end cap is in a closed position and deploy laterally from the longitudinal axis when switching into the open position;
the elongate member includes a rod connected to the distal tip of the end cap; and
the deployment mechanism includes a spring positioned about the elongate member, the spring preloaded to exert the threshold force on a first face of a retainer connected to the rod, where the threshold force exerted by the spring prevents the rod from translating in a direction away from the end cap and where the retainer includes a second face abutting a housing preventing translation of the rod in a direction toward the end cap,
wherein when a force on the distal end of the end cap exceeds the threshold force, the rod translates axially compressing the spring and thereby translating the distal end of the end cap causing the one or more fin members to deploy laterally switching the end cap into the open position.

25. The uterine length measuring device of claim 21, further comprising;
an indicator configured to provide an indication to a user when the end cap has switched from the closed to the open position.

26. A method for using a uterine measurement device, comprising:
transcervically inserting a uterine measurement device including advancing a first elongate member of the device to approximately the internal cervical os;
advancing a second elongate member of the device, located within the first elongate member, relative to the first elongate member to an internal fundus of the uterine cavity; and
measuring the uterine cavity length according to the position of the second elongate member relative to the first elongate member.

27. The method of claim 26, further comprising:
automatically locating the internal cervical os according to a feature positioned at the distal end of the first elongate member.

28. The method of claim 27, where the locating includes receiving tactile feedback from the feature contacting the internal cervical os.

29. The method of claim 27, where the feature is a step feature.

30. The method of claim 27, where the feature is a balloon feature.

31. The method of claim 27, where the feature is a groove feature.

32. The method of claim 26, further comprising:
sliding a control knob along the first elongate member to advance the second elongate member.

33. The method of claim 32, further comprising:
locking the control knob to hold the second elongate member in position relative to the first elongate member.

34. The method of claim 33, where locking the control knob comprises:
rotating the control knob into a groove in the first elongate member.

35. The method of claim 33, where locking the control knob comprises:
rotating a slidable collar attached to the control knob, where rotating the slidable collar tightens the collar around the first elongate member and holds the control knob in position.

36. The method of claim 26, further comprising:
moving an element along the first elongate member to the external cervical os to measure the cervical canal length according to the position of the movable element relative to the first elongate member.
